(19) United States
(12) Patent Application Publication
(10) Pub. No.: US 2026/0134977 A1
(43) Pub. Date: May 14, 2026

(54) ULTRASOUND DATA PROCESSING CONTROL APPARATUS AND ULTRASOUND DATA PROCESSING CONTROL PROGRAM

(71) Applicant: FUJIFILM Corporation, Tokyo (JP)

(72) Inventors: Tomoaki CHONO, Tokyo (JP); Hidenao KUBOTA, Tokyo (JP); Koji WAKI, Tokyo (JP)

(73) Assignee: FUJIFILM Corporation, Tokyo (JP)

(21) Appl. No.: 19/379,751

(22) Filed: Nov. 5, 2025

(30) Foreign Application Priority Data
Nov. 8, 2024 (JP) 2024-196173

(51) Int. Cl. G16H 30/20

(52) U.S. Cl. CPC G16H 30/20

(57) ABSTRACT

An ultrasound DB included in an ultrasound diagnostic apparatus stores ultrasound data to which a processing parameter is assigned. The ultrasound diagnostic apparatus acquires, from an image analysis apparatus including an external-apparatus-side processing module capable of executing processing equivalent to processing of an on-apparatus processing module, external apparatus information indicating whether or not the image analysis apparatus is permitted to execute processing equivalent to processing of the ultrasound data processing control apparatus on the ultrasound data. A transmission controller determines, based on the external apparatus information received in advance from the image analysis apparatus, content of information to be transmitted to the image analysis apparatus.

### CROSS-REFERENCE TO RELATED APPLICATION

This application claims the priority benefit of Japan application serial no. 2024-196173, filed on November 8, 2024. The entirety of the above-mentioned patent application is hereby incorporated by reference herein and made a part of this specification.

### BACKGROUND OF THE INVENTION

### 1. Field of the Invention

The present specification relates to an improvement of an ultrasound data processing control apparatus and an ultrasound data processing control program.

### 2. Description of the Related Art

In the related art, an ultrasound diagnostic apparatus that forms ultrasound data representing the inside of a subject based on reflected waves from the subject in a case in which ultrasonic waves are transmitted to the subject is known. In addition, in the related art, an ultrasound image processing apparatus that processes the ultrasound data formed by the ultrasound diagnostic apparatus is known. Examples of the ultrasound image processing apparatus include an ultrasound diagnostic apparatus itself and an image analysis apparatus that analyzes or manages the ultrasound data.

In particular, the ultrasound image processing apparatus that comprises various processing modules for executing various types of processing on the ultrasound data, selects a processing module to be used depending on an intended use, and causes the selected processing module to execute processing on the ultrasound data is known. In the present specification, the processing module means hardware alone or a combination of hardware and software for achieving a cohesive function.

For example, JP2003-271924A discloses a medical image processing apparatus that comprises a template in which a procedure of processing medical image data including ultrasound data is described, and a processing module for processing the medical image data, and that processes the medical image data by calling the processing module in accordance with the procedure described in the template.

### SUMMARY OF THE INVENTION

Meanwhile, an ultrasound data processing system including a plurality of ultrasound image processing apparatuses that process the ultrasound data is considered.

In the present specification, the term "ultrasound data" encompasses data obtained by transmitting and receiving the ultrasonic waves to and from the subject, reception beam data before being image-processed, or an ultrasound image formed based on the reception beam data. In addition, the ultrasound data may be two-dimensional (2D) ultrasound data or three-dimensional (3D) ultrasound data in which data indicating the signal intensity of the reflected wave from the subject is arranged in two or three dimensions, and may further be four-dimensional (4D) ultrasound data in which the 3D ultrasound data is arranged in a time direction. In addition, in the present specification, an ultrasound image processing apparatus that is included in the ultrasound data processing system and that transmits the ultrasound data to another ultrasound image processing apparatus will be referred to as an "ultrasound data processing control apparatus" (an apparatus according to an embodiment of the present invention), and one or a plurality of other ultrasound image processing apparatuses that are included in the ultrasound data processing system will be referred to as "external apparatuses". An administrator of the external apparatus may be the same as or different from an administrator of the ultrasound data processing control apparatus.

The ultrasound data processing control apparatus includes a processing module for processing the ultrasound data. In the present specification, the processing module included in the ultrasound data processing control apparatus will be referred to as an "on-apparatus processing module". As the on-apparatus processing module, a processing module developed by an external developer other than the administrator (for example, a manufacturer) of the ultrasound data processing control apparatus can be adopted. Similarly, the external apparatus also includes a processing module for processing the ultrasound data. In the present specification, the processing module included in the external apparatus will be referred to as an "external-apparatus-side processing module". As described above, since the external-apparatus-side processing module can be developed by the external developer, even in a case in which the administrator of the ultrasound data processing control apparatus and the administrator of the external apparatus are different from each other, the external-apparatus-side processing module included in the external apparatus can execute processing equivalent to processing of the on-apparatus processing module. It should be noted that, since the on-apparatus processing module is customized based on a request from the administrator of the ultrasound data processing control apparatus and the external-apparatus-side processing module is customized based on a request from the administrator of the external apparatus, the on-apparatus processing module and the external-apparatus-side processing module may not execute exactly the same processing, but since the bases thereof are the same, the two processing modules execute similar processing. In the present specification, the expression "execute equivalent processing" includes a case in which similar processing is executed.

A processing parameter for processing the ultrasound data may be assigned to the ultrasound data. As a result, the on-apparatus processing module can execute the processing on the ultrasound data using the processing parameter. In addition, since the external-apparatus-side processing module that executes the processing equivalent to the processing of the on-apparatus processing module is provided in the external apparatus, the external-apparatus-side processing module can execute the processing equivalent to the processing of the ultrasound data processing control apparatus on the ultrasound data using the processing parameter by transmitting, from the ultrasound data processing control apparatus to the external apparatus, the ultrasound data to which the processing parameter is assigned.

Here, from the viewpoint of the ultrasound data processing control apparatus, both cases can be considered: cases in which the external apparatus is permitted to use the processing parameter assigned to the ultrasound data and execute, on the ultrasound data, the processing equivalent to the processing of the ultrasound data processing control apparatus, and cases in which it is not. For example, the processing parameter assigned to the ultrasound data is a parameter specific to a certain company (here, company A) and is a parameter that will yield excellent processing results. In this case, for example, in a case in which the administrator of the ultrasound data processing control apparatus and the administrator of the external apparatus are the same as each other (for example, in a case in which company A manages both apparatuses), the ultrasound data to which the processing parameter is assigned may be transmitted to the external apparatus to cause the external apparatus to execute the processing equivalent to the processing of the ultrasound data processing control apparatus on the ultrasound data. On the other hand, in a case in which the administrator of the ultrasound data processing control apparatus and the administrator of the external apparatus are different from each other (for example, in a case in which company A manages the ultrasound data processing control apparatus and company B manages the external apparatus), from the viewpoint of preventing company-A-specific processing parameter from being transmitted to company B, the ultrasound data to which the processing parameter is assigned may be prohibited from being transmitted to the external apparatus, and the external apparatus may be prohibited from executing the processing equivalent to the processing of the ultrasound data processing control apparatus on the ultrasound data.

An object of the ultrasound data processing control apparatus disclosed in the present specification is to control whether or not an external apparatus is permitted to execute processing equivalent to processing of an ultrasound data processing control apparatus on ultrasound data.

The present specification discloses an ultrasound data processing control apparatus comprising: a processor; and a memory, in which the memory stores ultrasound data that is obtained by transmitting and receiving ultrasonic waves to and from a subject and to which a processing parameter for processing the ultrasound data is assigned, and the processor is configured to: function as an on-apparatus processing module that is included in the ultrasound data processing control apparatus and that processes the ultrasound data using the processing parameter; acquire, from an external apparatus including an external-apparatus-side processing module capable of executing processing equivalent to processing of the on-apparatus processing module, external apparatus information indicating whether or not the external apparatus is permitted to execute processing equivalent to processing of the ultrasound data processing control apparatus on the ultrasound data; and determine, based on the external apparatus information, content of information to be transmitted to the external apparatus in response to a transmission request for the ultrasound data from the external apparatus.

The processing parameters may include a first processing parameter for the on-apparatus processing module and a second processing parameter for the external-apparatus-side processing module, and the processor may be configured to, in a case in which the external apparatus information indicates that the external apparatus is permitted to execute the processing equivalent to the processing of the ultrasound data processing control apparatus on the ultrasound data, transmit the ultrasound data to which the first processing parameter is assigned, to the external apparatus.

The external apparatus information may further include information indicating content of processing executable by the external-apparatus-side processing module, and the processor may be configured to, in a case in which the external apparatus information indicates that the external-apparatus-side processing module is unable to execute specific processing that is processing executable by the on-apparatus processing module, further transmit a processing result of the specific processing executed by the on-apparatus processing module using the first processing parameter to the external apparatus.

The processor may be configured to, in a case in which the external apparatus information indicates that the external apparatus is not permitted to execute the processing equivalent to the processing of the ultrasound data processing control apparatus on the ultrasound data, transmit the ultrasound data to the external apparatus while prohibiting the processing executed by the external-apparatus-side processing module on the ultrasound data.

The processing parameters may include a first processing parameter for the on-apparatus processing module and a second processing parameter for the external-apparatus-side processing module, and the processor may be configured to, in a case in which the external apparatus information indicates that the external apparatus is not permitted to execute the processing equivalent to the processing of the ultrasound data processing control apparatus on the ultrasound data, transmit the ultrasound data to which the first processing parameter is not assigned and the second processing parameter is assigned, to the external apparatus.

The external apparatus information may further include information indicating content of processing executable by the external-apparatus-side processing module, and the processor may be configured to, in a case in which the external apparatus information indicates that the external-apparatus-side processing module is unable to execute specific processing that is processing executable by the on-apparatus processing module, further transmit a processing result of the specific processing executed by the on-apparatus processing module using the second processing parameter to the external apparatus.

The processor may be configured to assign the processing parameter to the ultrasound data.

The external apparatuses may include a first external apparatus that is permitted to execute the processing equivalent to the processing of the ultrasound data processing control apparatus and a second external apparatus that is not permitted to execute the processing equivalent to the processing of the ultrasound data processing control apparatus, and the processor may be configured to, based on the external apparatus information acquired from the first external apparatus and the external apparatus information acquired from the second external apparatus, transmit information with mutually different content to the first external apparatus and the second external apparatus.

The processor may be configured to acquire a processing result obtained by the external apparatus for the transmitted ultrasound data.

The present specification further discloses an ultrasound data processing control program causing a computer including a memory that stores ultrasound data that is obtained by transmitting and receiving ultrasonic waves to and from a subject and to which a processing parameter for processing the ultrasound data is assigned, to: function as an on-apparatus processing module that is included in an ultrasound data processing control apparatus and that processes the ultrasound data using the processing parameter; acquire, from an external apparatus including an external-apparatus-side processing module capable of executing processing equivalent to processing of the on-apparatus processing module, external apparatus information on the external apparatus; and determine, based on the external apparatus information, content of information to be transmitted to the external apparatus in response to a transmission request for the ultrasound data from the external apparatus.

With the ultrasound data processing control apparatus disclosed in the present specification, it is possible to control whether or not the external apparatus is permitted to execute the processing equivalent to the processing of the ultrasound data processing control apparatus on the ultrasound data.

### BRIEF DESCRIPTION OF THE DRAWINGS

FIG. 1 is a schematic configuration diagram of an ultrasound data processing system according to the present embodiment.

FIG. 2 is a schematic configuration diagram of a first image analysis apparatus.

FIG. 3 is a schematic configuration diagram of a second image analysis apparatus.

FIG. 4 is a schematic configuration diagram of an ultrasound diagnostic apparatus.

FIG. 5A is a first diagram showing an example of a reconstructed ultrasound image.

FIG. 5B is a second diagram showing an example of the reconstructed ultrasound image.

FIG. 5C is a third diagram showing an example of the reconstructed ultrasound image.

FIG. 6 is a flowchart showing a flow of processing by the ultrasound diagnostic apparatus.

### DESCRIPTION OF THE PREFERRED EMBODIMENTS

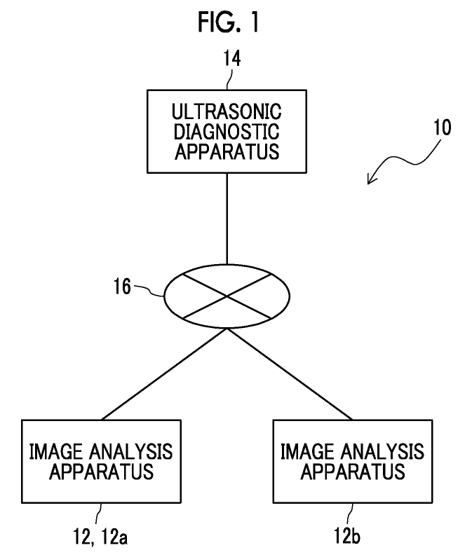

FIG. 1 is a schematic configuration diagram of an ultrasound data processing system 10 according to the present embodiment. The ultrasound data processing system 10 includes an image analysis apparatus 12 as an external apparatus and an ultrasound diagnostic apparatus 14 as an ultrasound data processing control apparatus. In the example in FIG. 1, two image analysis apparatuses 12 (image analysis apparatus 12a and image analysis apparatus 12b) are shown, but the ultrasound data processing system 10 may include only one image analysis apparatus 12 or may include three or more image analysis apparatuses 12. In the present specification, in a case in which the image analysis apparatuses 12a and 12b are not distinguished from each other, the image analysis apparatuses 12a and 12b will be simply referred to as the image analysis apparatus 12. Each image analysis apparatus 12 and the ultrasound diagnostic apparatus 14 are connected to each other so as to be communicable via a communication line 16 such as a wide area network (WAN) or a local area network (LAN).

Although the details will be described later, an outline of processing executed by the ultrasound data processing system 10 is as follows.

First, ultrasound data is formed in the ultrasound diagnostic apparatus 14. As described above, the term "ultrasound data" encompasses reception beam data and an ultrasound image, and encompasses 2D ultrasound data, 3D ultrasound image data, and 4D ultrasound data. In the present embodiment, the ultrasound data is 4D ultrasound data. In addition, the present embodiment is not limited thereto, but in the present embodiment, the ultrasonic waves are transmitted and received to and from the heart of a subject, and the 4D ultrasound data represents the heart.

A processing parameter for processing the ultrasound data is assigned to the ultrasound data. In the present embodiment, the processing parameter is assigned to the ultrasound data by the ultrasound diagnostic apparatus 14. The ultrasound diagnostic apparatus 14 includes an on-apparatus processing module. The on-apparatus processing module executes processing on the ultrasound data using the processing parameter assigned to the ultrasound data. In addition, the ultrasound diagnostic apparatus 14 can transmit the ultrasound data to which the processing parameter is assigned, to the image analysis apparatus 12.

The image analysis apparatus 12 includes an external-apparatus-side processing module that can execute processing equivalent to (having content equivalent to) the processing executed by the on-apparatus processing module of the ultrasound diagnostic apparatus 14. The external-apparatus-side processing module executes processing on the ultrasound data using the processing parameter assigned to the ultrasound data.

Although the outline of the processing executed by the ultrasound data processing system 10 is described as above, in the present embodiment, the ultrasound diagnostic apparatus 14 controls whether or not the image analysis apparatus 12 executes the processing equivalent to the processing of the ultrasound diagnostic apparatus 14 on the ultrasound data, by controlling the data transmitted to the image analysis apparatus 12.

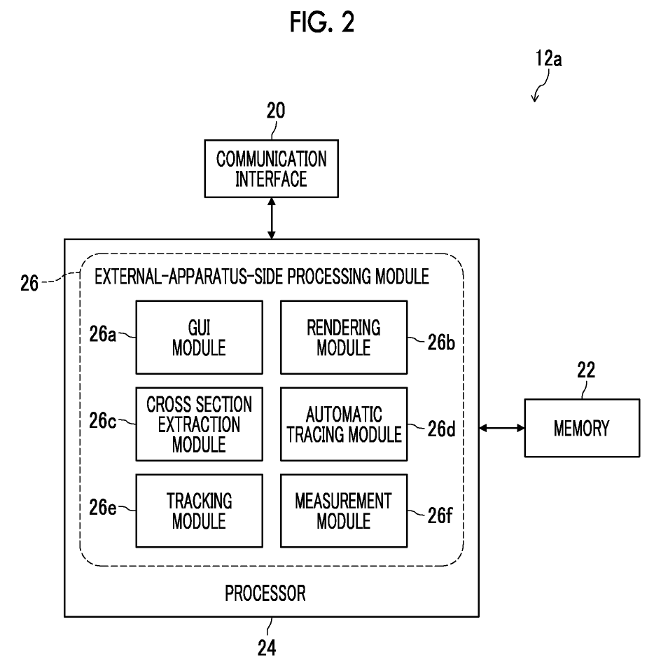

FIG. 2 is a schematic configuration diagram of the image analysis apparatus 12a. The image analysis apparatus 12a is an apparatus that provides the image analysis service of analyzing or managing the ultrasound data to a user. The image analysis apparatus 12a may be, for example, a server computer. In the present embodiment, it is assumed that an administrator of the image analysis apparatus 12a is the same as an administrator of the ultrasound diagnostic apparatus 14. For example, the image analysis apparatus 12a is a server computer managed by the administrator of the ultrasound diagnostic apparatus 14, and is a server computer for providing the image analysis service of the same administrator. The image analysis service can also analyze or manage medical image data acquired by various modalities (for example, a computed tomography (CT) apparatus or a magnetic resonance imaging (MRI) apparatus), in addition to the ultrasound data.

A communication interface 20 is configured as, for example, a network adapter. The communication interface 20 is configured to communicate with the ultrasound diagnostic apparatus 14 and a user terminal used by the user of the image analysis service via the communication line 16.

In particular, the communication interface 20 transmits, to the ultrasound diagnostic apparatus 14, external apparatus information indicating whether or not the image analysis apparatus 12a is permitted to execute the processing equivalent to the processing of the ultrasound diagnostic apparatus 14 on the ultrasound data. For example, the external apparatus information may be information indicating the administrator of the image analysis apparatus 12a. In the present embodiment, since the administrator of the image analysis apparatus 12a and the administrator of the ultrasound diagnostic apparatus 14 are the same as each other, the external apparatus information indicates the administrator of the image analysis apparatus 12a. Since the ultrasound diagnostic apparatus 14 has information indicating the administrator of the ultrasound diagnostic apparatus 14, in a case in which the administrator indicated by the external apparatus information received from the image analysis apparatus 12a is the same as the administrator of the ultrasound diagnostic apparatus 14, it is possible to determine that the image analysis apparatus 12a is permitted to execute the processing equivalent to the processing of the ultrasound diagnostic apparatus 14. On the other hand, in a case in which the administrator indicated by the external apparatus information received from the image analysis apparatus 12a is different from the administrator of the ultrasound diagnostic apparatus 14, the ultrasound diagnostic apparatus 14 can determine that the image analysis apparatus 12a is not permitted to execute the processing equivalent to the processing of the ultrasound diagnostic apparatus 14.

The external apparatus information is not limited to the information indicating the administrator of the image analysis apparatus 12a. For example, even in a case in which the administrator of the image analysis apparatus 12a (here, company B) and the administrator of the ultrasound diagnostic apparatus 14 (here, company A) are different from each other, in a case in which the company A and the company B have concluded a contract in which the company A permits the company B to execute the processing using the processing parameter of the company A, the image analysis apparatus 12a may be permitted to execute the processing equivalent to the processing of the ultrasound diagnostic apparatus 14 on the ultrasound data. In such a case, the external apparatus information can be information indicating contract content between the company A and the company B regarding the processing content of the ultrasound data.

The external apparatus information may further indicate processing content that can be executed by an external-apparatus-side processing module 26 (described later) of the image analysis apparatus 12a. For example, in the example of FIG. 2, since the external-apparatus-side processing module 26 functions as a GUI module 26a, a rendering module 26b, an automatic tracing module 26d, a tracking module 26e, and a measurement module 26f, the external apparatus information may include information indicating these processing modules.

In addition, the communication interface 20 receives the ultrasound data to which the processing parameter is assigned, from the ultrasound diagnostic apparatus 14. Further, the communication interface 20 transmits a result of the processing executed by the external-apparatus-side processing module 26 (described later) on the ultrasound data to the ultrasound diagnostic apparatus 14.

A memory 22 includes a hard disk drive (HDD), a solid state drive (SSD), an embedded Multi Media Card (eMMC), a read only memory (ROM), a random access memory (RAM), or the like. The memory 22 stores various types of medical data managed by the image analysis service and analysis results on the various types of medical data, in addition to the ultrasound data received from the ultrasound diagnostic apparatus 14.

The processor 24 executes various types of processing related to the image analysis service. Specifically, as shown in FIG. 2, the processor 24 functions as the external-apparatus-side processing module 26. In the present embodiment, the external-apparatus-side processing module 26 is composed of the GUI module 26a that executes processing related to a graphical user interface (GUI) provided to the user, the rendering module 26b that executes rendering processing on 3D or 4D ultrasound data to form a reconstructed ultrasound image that is a 2D ultrasound tomographic image, a cross section extraction module 26c that extracts a desired cross section from the ultrasound data, an automatic tracing module 26d that detects a feature point (feature region) in the ultrasound data, the tracking module 26e that tracks a temporal change in the position of the detected feature point, and the measurement module 26f that executes various types of measurement using the ultrasound data. That is, the external-apparatus-side processing module 26 can execute each type of processing. It should be noted that the processing that can be executed by the external-apparatus-side processing module 26 is not limited thereto.

The external-apparatus-side processing module 26 may be developed by an external developer other than the administrator of the image analysis apparatus 12a and the ultrasound diagnostic apparatus 14. In addition, the external-apparatus-side processing module 26 may be customized for the administrator of the image analysis apparatus 12a and the ultrasound diagnostic apparatus 14 in response to a request from the administrator.

The external-apparatus-side processing module 26 executes, using the processing parameter, processing on the ultrasound data transmitted together with the processing parameter received from the ultrasound diagnostic apparatus 14.

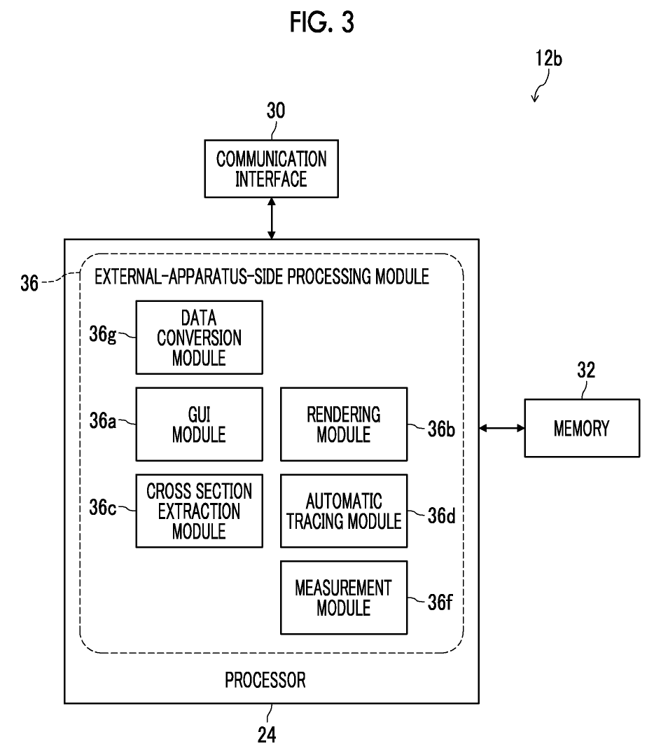

FIG. 3 is a schematic configuration diagram of the image analysis apparatus 12b. The image analysis apparatus 12b is an apparatus that provides the image analysis service to the user, like the image analysis apparatus 12a. The image analysis apparatus 12b may be, for example, a server computer. In the present embodiment, an administrator of the image analysis apparatus 12b is different from the administrator of the ultrasound diagnostic apparatus 14. For example, in a case in which the administrator of the ultrasound diagnostic apparatus 14 is the company A, the image analysis apparatus 12b is a server computer managed by the company B different from the company A and is a server computer for providing the image analysis service of the company B. Like the image analysis service provided by the image analysis apparatus 12a, the image analysis service provided by the image analysis apparatus 12b may also be capable of analyzing or managing the medical image data acquired in various modalities in addition to the ultrasound data.

The configurations and the functions of the communication interface 30 and a memory 32 are the same as the configurations and the functions of the communication interface 20 and the memory 22 of the image analysis apparatus 12a, and thus duplicated description will be omitted.

The processor 34 executes various types of processing related to the image analysis service. Specifically, as shown in FIG. 3, the processor 34 functions as an external-apparatus-side processing module 36. In the present embodiment, the external-apparatus-side processing module 36 includes a GUI module 36a, a rendering module 36b, a cross section extraction module 36c, an automatic tracing module 36d, a measurement module 36f, and a data conversion module 36g that converts the data format of the ultrasound data received from the ultrasound diagnostic apparatus 14. That is, the external-apparatus-side processing module 36 can execute each type of processing. It should be noted that the processing that can be executed by the external-apparatus-side processing module 36 is not limited thereto.

The external-apparatus-side processing module 36 may be developed by the external developer who has developed the external-apparatus-side processing module 26 of the image analysis apparatus 12a. Therefore, the content of the processing executed by the GUI module 36a, the rendering module 36b, the cross section extraction module 36c, the automatic tracing module 36d, and the measurement module 36f is equivalent to the content of the processing executed by the GUI module 26a, the rendering module 26b, the cross section extraction module 26c, the automatic tracing module 26d, and the measurement module 26f included in the external-apparatus-side processing module 26 of the image analysis apparatus 12a, respectively. Like the external-apparatus-side processing module 26, the external-apparatus-side processing module 36 may also be customized for the administrator of the image analysis apparatus 12b in response to a request from the administrator. Therefore, the external-apparatus-side processing module 26 and the external-apparatus-side processing module 36 may not execute exactly the same processing, but execute at least equivalent processing.

The external-apparatus-side processing module 36 of the image analysis apparatus 12b does not include the tracking module 26e included in the external-apparatus-side processing module 26 of the image analysis apparatus 12a, but includes the data conversion module 36g instead. As described above, the content of the processing that can be executed by the external-apparatus-side processing module 26 may be different between the image analysis apparatuses 12.

The external-apparatus-side processing module 36 executes, using the processing parameter, processing on the ultrasound data transmitted together with the processing parameter received from the ultrasound diagnostic apparatus 14. Since the administrator of the image analysis apparatus 12b and the administrator of the ultrasound diagnostic apparatus 14 are different from each other, it is required to convert (format) the ultrasound data transmitted from the ultrasound diagnostic apparatus 14 in order to process the ultrasound data by the external-apparatus-side processing module 36 of the image analysis apparatus 12b. The format information, which is information for converting the ultrasound data, is provided by the ultrasound diagnostic apparatus 14. The data conversion module 36g converts the ultrasound data based on the format information. The other processing modules included in the external-apparatus-side processing module 36 execute processing on the converted ultrasound data.

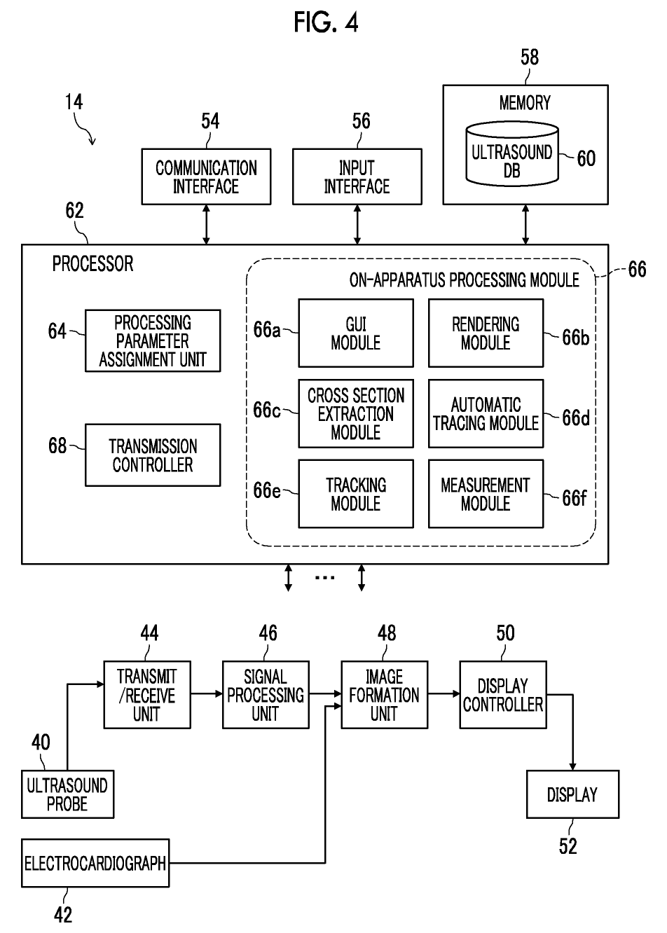

FIG. 4 is a schematic configuration diagram of the ultrasound diagnostic apparatus 14. The ultrasound diagnostic apparatus 14 is a medical apparatus installed in medical institutions such as a hospital.

An ultrasound probe 40 is a device that transmits and receives ultrasonic waves to and from the subject. The ultrasound probe 40 has a transducer element array consisting of a plurality of transducer elements that scan the subject with the ultrasonic beam. The ultrasound probe 40 may be a 1D probe in which a plurality of transducer elements are arranged in one row, or a 2D probe in which a plurality of transducer elements are arranged in a two-dimensional direction. A transmission signal is supplied to each transducer element from a transmit/receive unit 44 described later, so that each transducer element generates the ultrasonic beam that scans an ultrasound scanning plane. Each transducer element receives the reflected waves of the transmitted ultrasonic beam from the subject. In the present embodiment, the ultrasound probe 40 transmits and receives the ultrasonic waves to and from the heart of the subject.

An electrocardiograph 42 detects electrocardiographic data indicating the pulsation of the heart of the subject. For example, the electrocardiograph 42 includes an electrode, the electromotive force of the subject is measured by attaching the electrode to the vicinity of the heart of the subject, and the electrocardiographic data is formed based on the measured electromotive force. The electrocardiographic data detected by the electrocardiograph 42 is transmitted to an image formation unit 48.

The transmit/receive unit 44 transmits the transmission signal to the ultrasound probe 40 (specifically, each transducer element of the transducer element array) under the control of a processor 62 described later. The transmit/receive unit 44 also receives a reception signal from each transducer element that has received the reflected waves from the subject. The transmit/receive unit 44 includes an adder and a plurality of delay elements corresponding to the respective transducer elements, and executes, using the adder and the plurality of delay elements, phase‑aligned summation (delay-and-sum) processing in which the reception signals from the respective transducer elements are phase‑aligned and added. As a result, reception beam data is formed in which information indicating the signal intensity of the waves reflected from the subject is arranged in a depth direction of the subject.

The signal processing unit 46 executes various types of signal processing including filter processing of applying a bandpass filter, detection processing, and the like, on the reception beam data from the transmit/receive unit 44. As a result, the ultrasound data is obtained. In a case in which the ultrasound probe 40 is a 1D probe, 2D reception beam data (corresponding to one ultrasound tomographic image) are formed as a plurality of reception beam data corresponding to the ultrasound scanning plane. The signal processing unit 46 can form 3D reception beam data (ultrasound volume data) based on a plurality of 2D reception beam data arranged in a direction perpendicular to the ultrasound scanning plane. Furthermore, the signal processing unit 46 can form 4D reception beam data (4D ultrasound volume data) based on a plurality of ultrasound volume data formed over time. In a case in which the ultrasound probe 40 is a 2D probe, the transmit/receive unit 44 forms three-dimensional reception beam data, and thus the signal processing unit 46 can form ultrasound volume data and 4D ultrasound volume data by executing various types of signal processing on the reception beam data. As described above, in the present embodiment, since the ultrasound data is 4D ultrasound data, the signal processing unit 46 forms 4D ultrasound volume data as the 4D ultrasound data.

The image formation unit 48 forms the ultrasound image based on the reception beam data that has been signal-processed by the signal processing unit 46. For example, the image formation unit 48 can form a 2D ultrasound image (ultrasound tomographic image) indicating a cross section (particularly, an ultrasound transmission/reception surface corresponding to the 2D reception beam data) of the subject based on the 2D reception beam data. In addition, the image formation unit 48 forms an electrocardiographic waveform based on the electrocardiographic data received from the electrocardiograph 42. The electrocardiographic waveform is a graph representing the electrical activity of the heart.

A display controller 50 performs control to display various images including the ultrasound tomographic image formed by the image formation unit 48 on a display 52. In addition, the display controller 50 displays a processing result for the ultrasound data obtained by an on-apparatus processing module 66, which will be described later, on the display 52. In particular, in the present embodiment, since the ultrasound data is the 4D ultrasound data, the display controller 50 may display the electrocardiographic waveform together with a processing result for the 4D ultrasound data (for example, the reconstructed ultrasound image obtained by the rendering processing) and display a phase of a heartbeat corresponding to the processing result.

The display 52 is a display device configured, for example, by a liquid crystal display or an organic electroluminescence (EL) display.

The transmit/receive unit 44, the signal processing unit 46, the image formation unit 48, and the display controller 50 of the ultrasound diagnostic apparatus 14 are implemented by a processor (the processor 62 described later or a processor different from the processor 62).

A communication interface 54 is configured as, for example, a network adapter. The communication interface 54 is configured to communicate with the image analysis apparatus 12 via the communication line 16. In particular, the communication interface 54 acquires external apparatus information from the image analysis apparatus 12. In addition, the communication interface 54 transmits the ultrasound data to which the processing parameter is assigned, to the image analysis apparatus 12.

An input interface 56 is configured by, for example, a button, a trackball, and a touch panel. The input interface 56 is used to input an instruction of an operator, who uses the ultrasound diagnostic apparatus 14, to the ultrasound diagnostic apparatus 14.

The memory 58 includes an HDD, an SSD, an eMMC, a ROM, a RAM, or the like. The memory 58 stores an ultrasound data processing control program for operating the respective units of the ultrasound diagnostic apparatus 14. The ultrasound data processing control program can also be stored in a non-transitory computer-readable storage medium such as a Universal Serial Bus (USB) memory or a CD-ROM. The ultrasound diagnostic apparatus 14 can read the ultrasound data processing control program from such a storage medium and execute the read ultrasound data processing control program.

As shown in FIG. 4, the memory 58 stores an ultrasound database (DB) 60. The ultrasound data (in the present embodiment, 4D ultrasound volume data) formed by the ultrasound diagnostic apparatus 14 and the processing parameter for processing the ultrasound data are stored in the ultrasound DB 60 in association with each other. In the present embodiment, the processing parameters assigned to the ultrasound data are assigned by a processing parameter assignment unit 64 described later, but the processing parameters may be assigned to the ultrasound data by another method. For example, the processing parameter may be assigned to the ultrasound data by an apparatus other than the ultrasound diagnostic apparatus 14.

The processor 62 functions as the processing parameter assignment unit 64, the on-apparatus processing module 66, and a transmission controller 68 in accordance with the ultrasound data processing control program stored in the memory 58. Hereinafter, the functions of the respective units that are executed by the processor 62 will be described in detail.

The processing parameter assignment unit 64 assigns one or a plurality of processing parameters to the ultrasound data. The processing parameter assigned to the ultrasound data may be prepared in advance and stored in the memory 58, or the processing parameter assignment unit 64 may calculate the processing parameter or select the processing parameter from among the prepared processing parameter group in accordance with the content of the ultrasound data to assign the processing parameter to the ultrasound data.

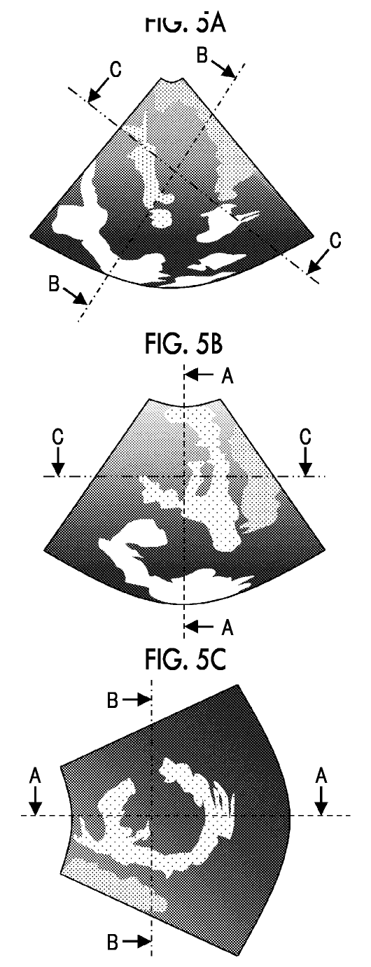

The processing parameters may include various parameters required for the on-apparatus processing module 66 or the external-apparatus-side processing modules 26 and 36 to execute the processing on the ultrasound data. In particular, in the present embodiment, since the ultrasound data is 4D ultrasound data, the processing parameters may include a processing parameter for processing the 4D ultrasound data. For example, a case will be considered in which the on-apparatus processing module 66 or the external-apparatus-side processing modules 26 and 36 execute the rendering processing on the 4D ultrasound data to form the reconstructed ultrasound image, and the reconstructed ultrasound image is displayed on the display 52. In this case, the specific processing parameter for processing the 4D ultrasound data includes position information of a line indicating a cutting plane of a multiplanar reconstruction (MPR) image (dashed line in FIG. 5B and FIG. 5C, one-dot chain line in FIG. 5A and FIG. 5C, and two-dot chain line in FIG. 5A and FIG. 5B) or a cross section position information obtained by cropping a rendering image.

The processing parameters may include a first processing parameter for the ultrasound diagnostic apparatus 14 (in other words, for the on-apparatus processing module 66 (described later)) and a second processing parameter for the image analysis apparatus 12 (in other words, for the external-apparatus-side processing modules 26 and 36) that is not permitted to execute the processing equivalent to the processing of the ultrasound diagnostic apparatus 14 on the ultrasound data. The first processing parameter is, for example, a processing parameter specific to a company that develops or manages the ultrasound diagnostic apparatus 14. The company can acquire the specific first processing parameter through its own research and development. By processing the ultrasound data using the specific first processing parameter, it is possible to obtain a favorable processing result (for example, high image quality, high accuracy, and the like) that cannot be obtained from other companies. Meanwhile, the second processing parameter is, for example, a widely known processing parameter. The second processing parameter is, at least, a processing parameter that does not obtain a favorable result in a case in which the ultrasound data is processed using the second processing parameter, as compared with the first processing parameter. Although the details will be described later, in a case in which the ultrasound data is stored in the ultrasound DB 60, the first processing parameter may be assigned to the ultrasound data, but the ultrasound data to which the second processing parameter is assigned instead of the first processing parameter may be transmitted to the image analysis apparatus 12 as necessary.

As a method of assigning the processing parameter to the ultrasound data, several methods can be considered. For example, in a case in which the ultrasound data conforms to the format defined in digital imaging and communications in medicine (DICOM), which is an international standard specification for medical images, the processing parameter assignment unit 64 can record the processing parameter in a data structure defined in DICOM. In the data structure defined in DICOM, each medical data is recorded with a tag number. The tags include a standardized tag (basic information such as a patient name and an examination time) and a private tag that can be freely defined, and the processing parameter assignment unit 64 can record the processing parameter in the private tag. Alternatively, the ultrasound data and the processing parameter may be in separate data files. In this case, the ultrasound data and the processing parameter are associated with each other by another method. For example, by giving the data file of the ultrasound data and the data file of the processing parameter the same name (but with different extensions), the ultrasound data and the processing parameter can be associated with each other.

The on-apparatus processing module 66 processes the ultrasound data using the processing parameter (particularly, the first processing parameter) assigned to the ultrasound data. In the present embodiment, the on-apparatus processing module 66 includes a GUI module 66a, a rendering module 66b, a cross section extraction module 66c, an automatic tracing module 66d, a tracking module 66e, and a measurement module 66f. As described above, the on-apparatus processing module 66 can execute a plurality of types of processing. The processing executed by the GUI module 66a, the rendering module 66b, the cross section extraction module 66c, the automatic tracing module 66d, the tracking module 66e, and the measurement module 66f is the same as the processing executed by the GUI module 26a, the rendering module 26b, the cross section extraction module 26c, the automatic tracing module 26d, the tracking module 26e, and the measurement module 26f included in the external-apparatus-side processing module 26 of the image analysis apparatus 12a, respectively. That is, the on-apparatus processing module 66 executes processing equivalent to the processing of the external-apparatus-side processing module 26. In the present embodiment, since the administrator of the image analysis apparatus 12a and the administrator of the ultrasound diagnostic apparatus 14 are the same as each other, the on-apparatus processing module 66 and the external-apparatus-side processing module 26 execute the processing of the same content.

On the other hand, since the administrator of the image analysis apparatus 12b and the administrator of the ultrasound diagnostic apparatus 14 are different from each other, the on-apparatus processing module 66 and the external-apparatus-side processing module 36 of the image analysis apparatus 12b may not execute processing of the same content due to customization by each administrator, but execute at least equivalent processing.

In addition, in a comparison between the external-apparatus-side processing module 36 and the on-apparatus processing module 66, there is a processing module that is included in the on-apparatus processing module 66 and that is not included in the external-apparatus-side processing module 36. In the examples of FIGS. 3 and 4, the tracking module 66e corresponds to the above-described processing module. That is, there is processing that can be executed by the on-apparatus processing module 66 but cannot be executed by the external-apparatus-side processing module 36. In the present specification, such processing will be referred to as "specific processing".

The on-apparatus processing module 66 executes processing on the ultrasound data stored in the ultrasound DB 60, using the first processing parameter assigned to the ultrasound data, based on the instruction from the operator of the ultrasound diagnostic apparatus 14.

The transmission controller 68 transmits the ultrasound data to the image analysis apparatus 12 based on the transmission request for the ultrasound data stored in the ultrasound DB 60 received from the image analysis apparatus 12. In particular, the transmission controller 68 determines, based on the external apparatus information received in advance from the image analysis apparatus 12, the content of the information to be transmitted to the image analysis apparatus 12. Specifically, the transmission controller 68 transmits different information to the image analysis apparatus 12 depending on whether or not the external apparatus information received from the image analysis apparatus 12 indicates that the image analysis apparatus 12 is permitted to execute the processing equivalent to the processing of the ultrasound diagnostic apparatus 14 on the ultrasound data.

First, a case will be described in which the transmission request for the ultrasound data is received from the image analysis apparatus 12 that is permitted to execute the processing equivalent to the processing of the ultrasound diagnostic apparatus 14 on the ultrasound data. In the present embodiment, since the image analysis apparatus 12a is permitted to execute the processing equivalent to the processing of the ultrasound diagnostic apparatus 14, a case will be described in which the transmission request for the ultrasound data is received from the image analysis apparatus 12a.

In a case in which the transmission request for the ultrasound data is received from the image analysis apparatus 12a, the transmission controller 68 refers to the external apparatus information received in advance from the image analysis apparatus 12a. As a result, the transmission controller 68 determines that the image analysis apparatus 12a (particularly, the external-apparatus-side processing module 26) is permitted to execute the processing equivalent to the processing of the ultrasound diagnostic apparatus 14 on the ultrasound data.

In this case, the transmission controller 68 transmits the ultrasound data which is indicated by the transmission request and to which the first processing parameter (that is, the processing parameter for the on-apparatus processing module 66) is assigned, to the image analysis apparatus 12a. As a result, the external-apparatus-side processing module 26 can execute processing on the ultrasound data using the first processing parameter. That is, the processing equivalent to the processing of the ultrasound diagnostic apparatus 14 can be executed on the ultrasound data.

In addition, a case will be considered in which the external-apparatus-side processing module 26 cannot execute the specific processing that can be executed by the on-apparatus processing module 66. The transmission controller 68 determines that the external-apparatus-side processing module 26 cannot execute the specific processing, based on the external apparatus information indicating the content of the processing executable by the external-apparatus-side processing module 26, which is received in advance from the image analysis apparatus 12a. In this case, since the image analysis apparatus 12a is permitted to execute the processing equivalent to the processing of the ultrasound diagnostic apparatus 14, the on-apparatus processing module 66 executes the specific processing on the ultrasound data related to the transmission request using the first processing parameter to obtain the processing result. Then, the transmission controller 68 transmits the processing result of the specific processing by the on-apparatus processing module 66 to the image analysis apparatus 12a together with the ultrasound data to which the first processing parameter is assigned. Accordingly, even in a case in which there is the specific processing that can be executed by the on-apparatus processing module 66 but cannot be executed by the external-apparatus-side processing module 26, the image analysis apparatus 12a can obtain a processing result equivalent to the processing result of the ultrasound diagnostic apparatus 14.

The processor 24 of the image analysis apparatus 12a may transmit the processing result obtained by the external-apparatus-side processing module 26 for the ultrasound data received from the ultrasound diagnostic apparatus 14 to the ultrasound diagnostic apparatus 14. That is, the processor 62 of the ultrasound diagnostic apparatus 14 may acquire the processing result of the image analysis apparatus 12a (that is, the external-apparatus-side processing module 26) on the ultrasound data. Setting aside a case in which the on-apparatus processing module 66 and the external-apparatus-side processing module 26 execute the same processing, in a case in which the on-apparatus processing module 66 and the external-apparatus-side processing module 26 execute different processing (although the processing is the equivalent processing), naturally, the processing result of the on-apparatus processing module 66 on the same ultrasound data and the processing result of the external-apparatus-side processing module 26 on the same ultrasound data are different from each other. In such a case, there is a case in which it is meaningful for the ultrasound diagnostic apparatus 14 to acquire the processing result obtained by the external-apparatus-side processing module 26 for the ultrasound data. For example, in a case in which the processing result of the on-apparatus processing module 66 can be used as training data of a learning model, an amount of training data acquired by the ultrasound diagnostic apparatus 14 can be increased by acquiring the processing result of the external-apparatus-side processing module 26.

Next, a case will be described in which the transmission request for the ultrasound data is received from the image analysis apparatus 12 that is not permitted to execute the processing equivalent to the processing of the ultrasound diagnostic apparatus 14 on the ultrasound data. In the present embodiment, since the image analysis apparatus 12b is not permitted to execute the processing equivalent to the processing of the ultrasound diagnostic apparatus 14, a case will be described in which the transmission request for the ultrasound data is received from the image analysis apparatus 12b.

In a case in which the transmission request for the ultrasound data is received from the image analysis apparatus 12b, the transmission controller 68 refers to the external apparatus information received in advance from the image analysis apparatus 12b. As a result, the transmission controller 68 determines that the image analysis apparatus 12b (particularly, the external-apparatus-side processing module 36) is not permitted to execute the processing equivalent to the processing of the ultrasound diagnostic apparatus 14 on the ultrasound data. There are several variations of the processing that can be executed by the transmission controller 68 in this case.

First, the transmission controller 68 can prevent the image analysis apparatus 12b from transmitting the ultrasound data for which the transmission request is made and the processing parameter assigned to the ultrasound data. As a result, naturally, the image analysis apparatus 12b cannot execute the processing on the ultrasound data. Therefore, the ultrasound diagnostic apparatus 14 can prevent the image analysis apparatus 12b from executing the processing equivalent to the processing of the ultrasound diagnostic apparatus 14 on the ultrasound data.

Second, the transmission controller 68 may transmit the ultrasound data to the image analysis apparatus 12b while prohibiting the external-apparatus-side processing module 36 from processing the ultrasound data. As a result, the image analysis apparatus 12b can store and manage the ultrasound data in the image analysis apparatus 12b, but cannot execute the processing on the ultrasound data. Therefore, the ultrasound diagnostic apparatus 14 can prevent the image analysis apparatus 12b from executing the processing equivalent to the processing of the ultrasound diagnostic apparatus 14 on the ultrasound data.

As the method of prohibiting the processing of the ultrasound data, various methods are considered. For example, the transmission controller 68 may transmit information for prohibiting the processing on the ultrasound data to the image analysis apparatus 12b together with the ultrasound data. As the information, for example, a processing prohibition flag for prohibiting the processing on the ultrasound data, which is assigned to the ultrasound data, may be used.

As described above, in a case in which the administrator of the image analysis apparatus 12b and the administrator of the ultrasound diagnostic apparatus 14 are different from each other, the ultrasound data may need to be converted before the external-apparatus-side processing module 36 processes the ultrasound data. The format information for converting the ultrasound data is provided by the ultrasound diagnostic apparatus 14. Therefore, the transmission controller 68 may transmit the ultrasound data to the image analysis apparatus 12b, but need not transmit the format information to the image analysis apparatus 12b. As a result, the external-apparatus-side processing module 36 (specifically, the data conversion module 36g) cannot convert the ultrasound data, and thus the image analysis apparatus 12b can prevent the processing from being executed on the ultrasound data.

Third, the transmission controller 68 may transmit the ultrasound data indicated by the transmission request, that is, the ultrasound data to which the first processing parameter is not assigned and the second processing parameter (that is, the processing parameter for the external-apparatus-side processing module 36) is assigned, to the image analysis apparatus 12b. Accordingly, the external-apparatus-side processing module 36 can execute the processing on the ultrasound data using the second processing parameter. In other words, although the external-apparatus-side processing module 36 cannot obtain results as good as those of processing equivalent to the processing of the ultrasound diagnostic apparatus 14 using the first processing parameter, the external-apparatus-side processing module 36 can execute at least some processing on the ultrasound data using the second processing parameter.

In addition, a case will be considered in which the external-apparatus-side processing module 36 cannot execute the specific processing that can be executed by the on-apparatus processing module 66. The transmission controller 68 determines that the external-apparatus-side processing module 36 cannot execute the specific processing, based on the external apparatus information indicating the content of the processing executable by the external-apparatus-side processing module 36, which is received in advance from the image analysis apparatus 12b. In the present embodiment, the on-apparatus processing module 66 includes the tracking module 66e, but the external-apparatus-side processing module 36 does not include the tracking module, so that the processing executed by the tracking module 66e is the specific processing. In this case, since the image analysis apparatus 12b is not permitted to execute the processing equivalent to the processing of the ultrasound diagnostic apparatus 14, the tracking module 66e executes the specific processing on the ultrasound data related to the transmission request using the second processing parameter, and obtains the processing result. The transmission controller 68 transmits the processing result of the specific processing by the tracking module 66e to the image analysis apparatus 12b together with the ultrasound data to which the second processing parameter is assigned. Accordingly, even in a case in which there is the specific processing that can be executed by the on-apparatus processing module 66 but cannot be executed by the external-apparatus-side processing module 36, the image analysis apparatus 12b can obtain the processing result of the processing on the ultrasound data using the second processing parameter.

In a case in which the external-apparatus-side processing module 36 cannot execute the specific processing, the transmission controller 68 may determine whether or not to transmit the processing result of the specific processing by the on-apparatus processing module 66 to the image analysis apparatus 12b based on the content of the specific processing. For example, in a case in which the value of the specific processing is high (for example, the specific processing is a technology that is important for the business strategy of the company A that manages the ultrasound diagnostic apparatus 14) or in a case in which the data capacity of the processing result of the specific processing is very large, the transmission controller 68 may not transmit the result of the specific processing to the image analysis apparatus 12b even in a case in which the external-apparatus-side processing module 36 cannot execute the specific processing. For example, as in the present embodiment, in a case in which the ultrasound data is the 4D ultrasound data, the transmission controller 68 may transmit the result of the specific processing to the image analysis apparatus 12b in a case in which the specific processing is the 2D analysis, but need not transmit the result of the specific processing to the image analysis apparatus 12b in a case in which the specific processing is the analysis of the 3D volume, the analysis based on the time change of the 4D, or the like.

The processor 34 of the image analysis apparatus 12b may transmit the processing result obtained by the external-apparatus-side processing module 36 for the ultrasound data received from the ultrasound diagnostic apparatus 14 to the ultrasound diagnostic apparatus 14. That is, the processor 62 of the ultrasound diagnostic apparatus 14 may acquire the processing result of the image analysis apparatus 12b (that is, the external-apparatus-side processing module 36) on the ultrasound data.

As described above, the transmission controller 68 transmits different information to the image analysis apparatus 12 depending on whether or not the external apparatus information received from the image analysis apparatus 12 indicates that the image analysis apparatus 12 is permitted to execute the processing equivalent to the processing of the ultrasound diagnostic apparatus 14 on the ultrasound data. As in the ultrasound data processing system 10 (see FIG. 1) according to the present embodiment, in a case in which the external apparatus includes the image analysis apparatus 12a as a first external apparatus that is permitted to execute the processing equivalent to the processing of the ultrasound diagnostic apparatus 14 and the image analysis apparatus 12b as a second external apparatus that is not permitted to execute the processing equivalent to the processing of the ultrasound diagnostic apparatus 14, the transmission controller 68 can transmit information with different content to the image analysis apparatus 12a and the image analysis apparatus 12b based on the external apparatus information acquired from the image analysis apparatus 12a and the image analysis apparatus 12b, respectively.

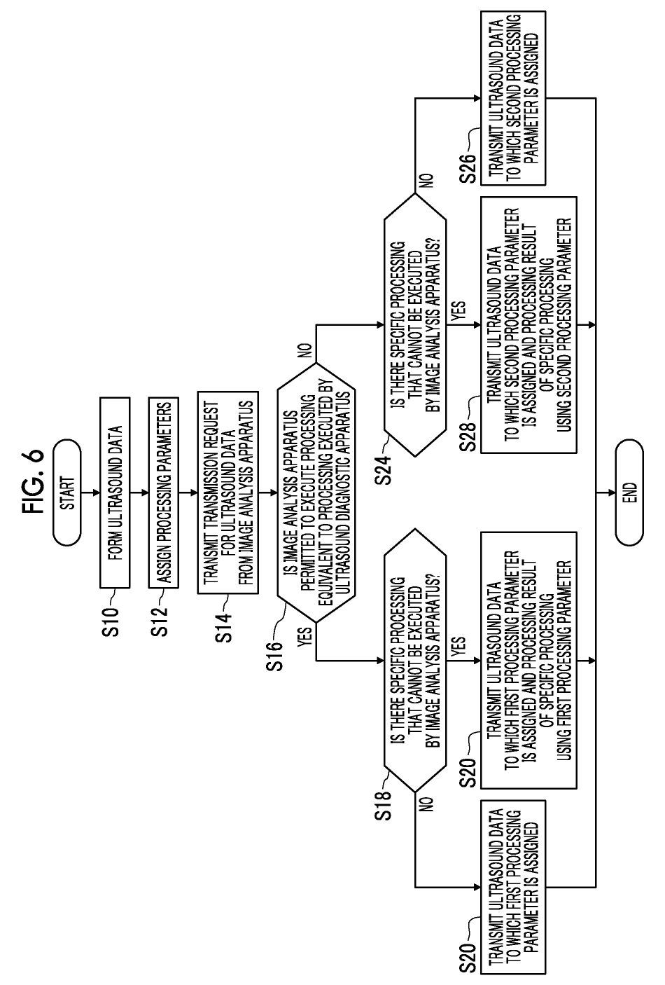

The outline of the ultrasound data processing system 10 according to the present embodiment has been described above. Hereinafter, a flow of the processing by the ultrasound diagnostic apparatus 14 will be described with reference to a flowchart shown in FIG. 6. It is assumed that at the start of the flowchart in FIG. 6, the ultrasound diagnostic apparatus 14 has already received the external apparatus information from each image analysis apparatus.

In step S10, the signal processing unit 46 forms the ultrasound data based on the reception signal obtained by transmitting and receiving the ultrasonic waves to and from the subject using the ultrasound probe 40. The formed ultrasound data is stored in the ultrasound DB 60.

In step S12, the processing parameter assignment unit 64 assigns the processing parameter to the ultrasound data formed in step S10. The processing parameters include the first processing parameter for the ultrasound diagnostic apparatus 14 and the second processing parameter for the image analysis apparatus 12 that is not permitted to execute the processing equivalent to the processing of the ultrasound diagnostic apparatus 14 on the ultrasound data.

In step S14, the ultrasound diagnostic apparatus 14 receives the transmission request for the ultrasound data from the image analysis apparatus 12.

In step S16, the transmission controller 68 determines whether or not the image analysis apparatus 12 is permitted to execute the processing equivalent to the processing of the ultrasound diagnostic apparatus 14 on the ultrasound data based on the external apparatus information received from the image analysis apparatus 12 that is the transmission request source of the ultrasound data.

In a case in which the image analysis apparatus 12 is permitted to execute the processing equivalent to the processing of the ultrasound diagnostic apparatus 14 on the ultrasound data, the processing proceeds to step S18, and in step S18, the transmission controller 68 determines whether or not there is the specific processing that cannot be executed by the external-apparatus-side processing modules 26 and 36 of the image analysis apparatus 12 but can be executed by the on-apparatus processing module 66 of the ultrasound diagnostic apparatus 14, based on the external apparatus information received from the image analysis apparatus 12 that is the transmission request source of the ultrasound data.

In a case in which there is no specific processing, the processing proceeds to step S20, and in step S20, the transmission controller 68 transmits the ultrasound data which is the ultrasound data indicated by the transmission request and to which the first processing parameter is assigned, to the image analysis apparatus 12.

In a case in which there is the specific processing, the processing proceeds to step S22, and in step S22, the transmission controller 68 transmits, to the image analysis apparatus 12, the ultrasound data to which the first processing parameter is assigned and the processing result of the specific processing executed by the on-apparatus processing module 66 using the first processing parameter.

The processing returns to step S16, and in a case in which the image analysis apparatus 12 is not permitted to execute the processing equivalent to the processing of the ultrasound diagnostic apparatus 14 on the ultrasound data, the processing proceeds to step S24.

In step S24, as in step S18, the transmission controller 68 determines whether or not there is the specific processing that cannot be executed by the external-apparatus-side processing modules 26 and 36 of the image analysis apparatus 12 but can be executed by the on-apparatus processing module 66 of the ultrasound diagnostic apparatus 14, based on the external apparatus information received from the image analysis apparatus 12 that is the transmission request source of the ultrasound data.

In a case in which there is no specific processing, the processing proceeds to step S26, and in step S26, the transmission controller 68 transmits the ultrasound data which is the ultrasound data indicated by the transmission request and to which the second processing parameter is assigned, to the image analysis apparatus 12.

In a case in which there is the specific processing, the processing proceeds to step S28, and in step S28, the transmission controller 68 transmits, to the image analysis apparatus 12, the ultrasound data to which the second processing parameter is assigned and the processing result of the specific processing executed by the on-apparatus processing module 66 using the second processing parameter.

Although not shown in FIG. 6, in step S16, in a case in which the image analysis apparatus 12 is not permitted to execute the processing equivalent to the processing of the ultrasound diagnostic apparatus 14 on the ultrasound data, the transmission controller 68 may not transmit the ultrasound data to the image analysis apparatus 12, or may transmit the ultrasound data to the image analysis apparatus 12 while prohibiting the processing of the ultrasound data by the external-apparatus-side processing modules 26 and 36.

Although the ultrasound data processing control apparatus according to the present disclosure has been described above, the ultrasound data processing control apparatus according to the present disclosure is not limited to the above-described embodiment, and various changes can be made without departing from the gist of the present disclosure.

For example, in the embodiment described above, the ultrasound data processing control apparatus is the ultrasound diagnostic apparatus 14, but the ultrasound data processing control apparatus may be any computer as long as the ultrasound DB 60 that stores the ultrasound data to which the processing parameter is assigned, the on-apparatus processing module 66, and the transmission controller 68 are included. In addition, the function of the ultrasound data processing control apparatus may be exhibited by cooperation of a plurality of apparatuses. In this case, the plurality of apparatuses correspond to the ultrasound data processing control apparatus.

In the present embodiment, each type of processing is executed by any computer. In addition, any computer may execute these types of processing by a processor as hardware, a program as software, or a combination thereof. In such a case, the processor is configured to execute various types of processing in the present embodiment in cooperation with the program, and may function as each unit or each means in the present embodiment. In addition, the execution order of the processing by the processor is not limited to the above-described order and may be changed as appropriate. Any computer may be a general-purpose computer, a computer for specific use, a workstation, or another system that can execute each type of processing.

The processor may be configured by one or more types of hardware, and the type of hardware is not limited. For example, the processor may be implemented by hardware such as a programmable logic device, for example, a central processing unit (CPU), a micro processing unit (MPU), or a field programmable gate array (FPGA), a dedicated circuit for executing specific processing such as an application specific integrated circuit (ASIC), a graphics processing unit (GPU), or a neural processing unit (NPU). Moreover, the kind of hardware may be a combination of different types of hardware. In a case in which the plurality of types of hardware are configured to execute one or a plurality of types of processing of a certain processor, the plurality of types of hardware may be present in devices physically separated from each other or may be present in the same device. Furthermore, in any of the embodiments, the order of each type of processing executed by the processor is not limited to the above-described order, and may be changed as appropriate. In addition, hardware is implemented in a form of an electric circuit (circuitry) in which circuit elements, such as semiconductor elements, are combined.

Furthermore, the program may be software such as firmware or microcode. The program may be, for example, a group of program modules, and each function thereof may be implemented by a processor configured to execute each function. The program may be a program code or a plurality of code segments stored in one or more non-transitory computer-readable media (for example, a storage medium and other storages). The program may be stored in the plurality of non-transitory computer-readable media present in devices physically separated from each other. The program code or the code segment may represent any combination of procedures, functions, subprograms, routines, subroutines, modules, software packages, classes, instructions, data structures, or program statements. The program code or the code segment may be connected to another code segment or a hardware circuit by transmitting and receiving information, data, arguments, parameters, or contents in the memory.

The present invention is also applicable to a program and a program product.

What is claimed is:

1. An ultrasound data processing control apparatus comprising:
a processor; and
a memory,
wherein the memory stores ultrasound data that is obtained by transmitting and receiving ultrasonic waves to and from a subject and to which a processing parameter for processing the ultrasound data is assigned, and
the processor is configured to:
function as an on-apparatus processing module that is included in the ultrasound data processing control apparatus and that processes the ultrasound data using the processing parameter;
acquire, from an external apparatus including an external-apparatus-side processing module capable of executing processing equivalent to processing of the on-apparatus processing module, external apparatus information indicating whether or not the external apparatus is permitted to execute processing equivalent to processing of the ultrasound data processing control apparatus on the ultrasound data; and
determine, based on the external apparatus information, content of information to be transmitted to the external apparatus in response to a transmission request for the ultrasound data from the external apparatus.

2. The ultrasound data processing control apparatus according to claim 1,
wherein the processing parameter includes a first processing parameter for the on-apparatus processing module and a second processing parameter for the external-apparatus-side processing module, and
the processor is configured to, in a case in which the external apparatus information indicates that the external apparatus is permitted to execute the processing equivalent to the processing of the ultrasound data processing control apparatus on the ultrasound data, transmit the ultrasound data to which the first processing parameter is assigned, to the external apparatus.

3. The ultrasound data processing control apparatus according to claim 2,
wherein the external apparatus information further includes information indicating content of processing executable by the external-apparatus-side processing module, and
the processor is configured to, in a case in which the external apparatus information indicates that the external-apparatus-side processing module is unable to execute specific processing that is processing executable by the on-apparatus processing module, further transmit a processing result of the specific processing executed by the on-apparatus processing module using the first processing parameter to the external apparatus.

4. The ultrasound data processing control apparatus according to claim 1,
wherein the processor is configured to, in a case in which the external apparatus information indicates that the external apparatus is not permitted to execute the processing equivalent to the processing of the ultrasound data processing control apparatus on the ultrasound data, transmit the ultrasound data to the external apparatus while prohibiting the processing executed by the external-apparatus-side processing module on the ultrasound data.

5. The ultrasound data processing control apparatus according to claim 1,
wherein the processing parameter includes a first processing parameter for the on-apparatus processing module and a second processing parameter for the external-apparatus-side processing module, and
the processor is configured to, in a case in which the external apparatus information indicates that the external apparatus is not permitted to execute the processing equivalent to the processing of the ultrasound data processing control apparatus on the ultrasound data, transmit the ultrasound data to which the first processing parameter is not assigned and the second processing parameter is assigned, to the external apparatus.

6. The ultrasound data processing control apparatus according to claim 5,
wherein the external apparatus information further includes information indicating content of processing executable by the external-apparatus-side processing module, and
the processor is configured to, in a case in which the external apparatus information indicates that the external-apparatus-side processing module is unable to execute specific processing that is processing executable by the on-apparatus processing module, further transmit a processing result of the specific processing executed by the on-apparatus processing module using the second processing parameter to the external apparatus.

7. The ultrasound data processing control apparatus according to claim 1,
wherein the processor is configured to assign the processing parameter to the ultrasound data.

8. The ultrasound data processing control apparatus according to claim 1,
wherein the external apparatus includes a first external apparatus that is permitted to execute the processing equivalent to the processing of the ultrasound data processing control apparatus and a second external apparatus that is not permitted to execute the processing equivalent to the processing of the ultrasound data processing control apparatus, and
the processor is configured to, based on the external apparatus information acquired from the first external apparatus and the external apparatus information acquired from the second external apparatus, transmit information with mutually different content to the first external apparatus and the second external apparatus.

9. The ultrasound data processing control apparatus according to claim 1,
wherein the processor is configured to acquire a processing result obtained by the external apparatus for the transmitted ultrasound data.

10. A non-transitory computer-readable storage medium storing an ultrasound data processing control program causing a computer including a memory that stores ultrasound data that is obtained by transmitting and receiving ultrasonic waves to and from a subject and to which a processing parameter for processing the ultrasound data is assigned, to:
function as an on-apparatus processing module that is included in an ultrasound data processing control apparatus and that processes the ultrasound data using the processing parameter;
acquire, from an external apparatus including an external-apparatus-side processing module capable of executing processing equivalent to processing of the on-apparatus processing module, external apparatus information indicating whether or not the external apparatus is permitted to execute processing equivalent to processing of the ultrasound data processing control apparatus on the ultrasound data; and
determine, based on the external apparatus information, content of information to be transmitted to the external apparatus in response to a transmission request for the ultrasound data from the external apparatus.
